(19) United States
(12) Patent Application Publication
(10) Pub. No.: US 2020/0148987 A1
(43) Pub. Date: May 14, 2020

(54) APPARATUS, SYSTEM AND METHOD FOR BIOPROCESSING

(71) Applicant: GE HEALTHCARE BIO-SCIENCES CORP., Marlborough, MA (US)

(72) Inventors: REGINALD DONOVAN SMITH, Schenectady, NY (US); Chengkun Zhang, Niskayuna, NY (US); Jason Castle, Niskasyuna, NY (US); Kenneth Conway, Niskayuna, NY (US)

(73) Assignee: GE HEALTHCARE BIO-SCIENCES CORP., Marlborough, MA (US)

(21) Appl. No.: 16/189,616

(22) Filed: Nov. 13, 2018

(51) Int. Cl. C12M 1/00

(52) U.S. Cl. CPC C12M 1/007; C12M 23/22; C12M 23/26; C12M 23/14

(57) ABSTRACT

A bioreactor vessel includes a body having upper and lowerends and a hollow interior cavity formed in the body, the interior cavity located between the upper and lower ends, the interior cavity being configured to receive biomaterials for processing. The interior cavity includes a lower boundary that is angled toward the lower end of the body such that the vessel may be tilted to allow biomaterials within the interior cavity to be extracted and. concentrated and/or washed without the need for a separate bioprocessing device.

### BACKGROUND

### Technical Field

Embodiments of the invention relate generally to bioprocessing and more specifically to an apparatus, system and method that facilitates the manufacture of cell therapies by combining multiple manufacturing processes in a single device.

### Discussion of Art

Various medical therapies involve the culture and expansion of cells to increase cell density for downstream therapeutic processes. For example, chimeric antigen receptor cell therapy, e.g., CAR-T, involves extraction of white blood cells from a donor and genetically engineering the cells in such a way that enables the cells to identify and attack malignant cells. Once engineered, the cells are transferred to cell culture/expansion vessels to allow the cells to proliferate to achieve a particular target dosage.

Many such therapies are manufactured utilizing equipment that provides agitation, temperature control and gas control, such as a rocking platform bioreactor, to activate extracted cells and expand them to a desired density. During these processes, cells may require washing to reduce impurities, e.g., remnants of a viral vector. Washing, however, typically requires the transfer of cells from the initial cell culture/expansion vessel to a separate, designated cell washing device. Once washed, the cells are then transferred to another cell culture vessel for additional processing.

Furthermore, it is often desirable to concentrate cells, i.e., reduce the volume of liquid without removing cells, during cell processing. More specifically, by reducing the volume of liquid prior to cell washing, the amount of wash buffer required and the amount of time necessary for washing, will also be reduced. As with washing, however, cell concentration typically requires the transfer of cells front the initial cell culture/expansion vessel to a separate device, e.g., a centrifuge, to reduce volume.

In addition to requiring multiple bioprocessing devices, many known bioreactor systems also require extensive fluid line handling to assemble and operate. That is, multiple individual fluid lines must be routed and connected to a plurality of pumps, bags and/or filters by an operator. As will be appreciated, it is generally desirable to reduce the amount of human interaction necessary to perform cell processing to provide an ease of manufacture and to reduce the possibility of any potential errors associated therewith.

In view of the above, there is a need for apparatus, systems and methods of cell processing that eliminate the need to transfer cells to a separate, designated device for concentration and/or washing, thereby reducing human intervention and providing an ease of manufacture.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible embodiments. Indeed, the disclosure may encompass a variety of forms that may be similar to or different from the embodiments set forth below.

In an embodiment, a bioreactor vessel includes a body having upper and lower ends and a hollow interior cavity formed in the body, the interior cavity located between the upper and lower ends, the interior cavity being configured to receive biomaterials for processing. The interior cavity includes a lower boundary that is angled toward the lower end of the body such that the vessel may be tilted to allow biomaterials within the interior cavity to be extracted and concentrated and/or washed without the need for a separate bioprocessing device.

In another embodiment, a bioprocessing system includes a bioreactor vessel having an interior cavity with a lower boundary at an angle of about 45 to about 75 degrees, the vessel configured for use with a tiltable bioreactor platform. The system further includes a pump mounting plate configured to engage a plurality of peristaltic pumps such that a plurality of fluid lines connectable to the bioreactor vessel operatively contact pump heads. Wherein upon tilting the bioreactor vessel to a substantially upright position, cells may be extracted from the bioreactor vessel and concentrated and/or washed, with the aid of the peristaltic pumps and an inline tangential flow filter, such that the cell concentration and/or washing can be accomplished without the need for a separate bioprocessing device.

In yet another embodiment, a method of bioprocessing includes processing cells in a bioreactor vessel having an interior cavity with a lower boundary at an angle of about 45 to about 75 degrees, tilting the bioreactor vessel to substantially upright position so that the cells may be extracted from the vessel, extracting cells from the bioreactor vessel, concentrating and/or washing the extracted cells. Wherein the steps of concentrating and/or washing the extracted cells are carried out without the need for a separate bioprocessing device.

In another embodiment, a pump mounting plate for operatively connecting a bioreactor vessel to at least one peristaltic pump includes at least one aperture through which a peristaltic pump can extend facilitating installation of the pump mounting plate to the peristaltic pump and a plurality of pump loop brackets configured to receive fluid lines connected to the vessel and position the fluid lines so that they are in operative contact with pump heads of the peristaltic pumps. The mounting plate further includes a filter bracket configured to removably secure a filter to the pump mounting plate. The pump mounting plate is configured to be preloaded with fluid lines, to provide an ease of installation and use of the vessel for cell processing.

### DRAWINGS

The present invention will be better understood from reading the following description of non-limiting embodiments, with reference to the attached drawings, wherein below:

FIG. 1 is a depiction of a bioprocessing system assembled on a wave bioreactor platform, according to an embodiment of the invention;

FIG. 2 depicts the bioprocessing system of FIG. 1 without the wave bioreactor platform and with the addition of a waste bag;

FIG. 3 is a graphical illustration of a bioreactor vessel configured for use with the bioprocessing system of FIG. 1;

FIG. 4 is a graphical illustration of a pump mounting plate configured for use with the bioprocessing system of FIG. 1;

FIGS. 5A-5E depict a process of securing a mounting plate to a plurality of peristaltic pumps according to an embodiment of the invention;

FIG. 6 is a schematic diagram of a bioprocessing system according to an embodiment of the invention; and

FIG. 7 is a graph illustrating the efficacy of bioprocessing systems according to embodiments of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts, without duplicative description.

As used herein, an element or step recited in the singular and proceeded with the word “a”, “an”, “the”, or “said” should be understood as not excluding plural of said elements or steps, unless such exclusion is explicitly stated. Furthermore, references to “one embodiment” or “an embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

As used herein, the terms “substantially,” “generally,” and “about” indicate conditions within reasonably achievable manufacturing and assembly tolerances, relative to ideal desired conditions suitable for achieving the functional purpose of a component or assembly.

As used herein, “bioreactor vessel” includes disposable and non-disposable plastic ware, bags and/or containers configured to receive biomaterials for bioprocessing, e.g., cell culture. The term includes single-use plastic ware, bags and/or containers and multiple-use plastic ware, bags and/or containers.

As used herein, a “bioprocessing device” refers to an apparatus, device, kit, or assembly, suitable for processing biomaterials, e.g., expanding, concentrating and/or washing cells. Such devices include, but are not limited to, bioreactors, bioreactor vessels, centrifuges, wash kits, filters and the like.

As used herein, a “closed system,” “closed bioreactor system,” or “closed bioprocessing system” refers to cell culture/bioreactor systems, vessels and accessory components that have been pre-sterilized while closed and/or sealed and that retain integrity and/or sterility. The systems, vessels and components are utilized without breach of the integrity of the system, permit fluid transfers in and/or out while maintaining asepsis, and are connectable to other closed systems without loss of integrity. A closed system bioreactor and/or vessel refers to a system in which cells, cell culture medium, chemicals and reagents are aseptically added, removed and/or manipulated without breach of integrity of the system (e.g., by opening the cap of a tube or lifting the lid off a cell culture plate or dish). Single-use or multiple-use bags and/or containers and/or bioreactors in a closed system are added onto or into the closed system for example by sterile tube welding at the site of the vessel or bioreactor.

A “separate bioprocessing device,” as used herein, refers to a bioprocessing device that is separate from, e.g., not connected to, or otherwise a part of, a closed bioreactor/bioprocessing system or a bioreactor vessel that is a component of a closed bioreactor/bioprocessing system.

By way of background, CAR-T involves extracting white blood cells from a subject, e.g., a human, or more generally a vertebrate, and genetically engineering them in such a way so they can identify and attack malignant cells. Typical CAR-T upstream processes include enrichment of peripheral blood mononuclear cells, isolation of the T-cells, activation, and transduction before a final expansion phase. For this latter phase, engineered. CAR-T cells are transferred to expansion devices to allow them to proliferate to meet a dose target. Cell culturing/processing devices for CART cells include a WAVE bioreactor, which constantly agitates the cell culture by creating a wave motion, while actively flowing gas and the option of exchanging cell culture media.

CAR-T processes typically require the transfer of T cells from an initial cell culture/expansion vessel to separate, designated devices for cell concentration and washing to remove, for example, remnants of a viral vector used in transduction. Moreover, many known cell culturing/processing devices also require extensive fluid line handling to assemble and operate. Embodiments of the present invention provide an easy to use, closed bioprocessing system in which cells may be concentrated and/or washed without the need for a separate bioprocessing device.

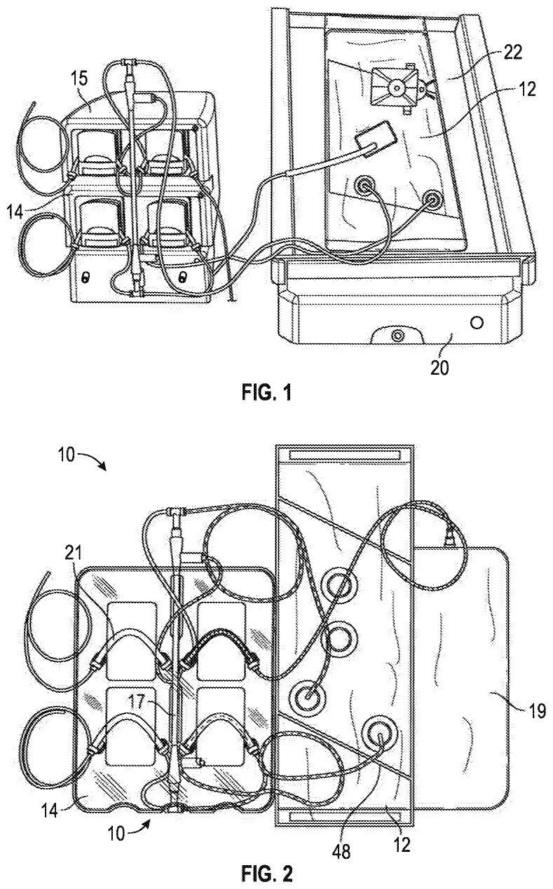

Referring now to FIGS. 1 and 2, components of a bioprocessing system 10, according to an embodiment of the invention, are shown installed on a bioreactor platform 20. In particular, the system 10 generally includes a fluidly coupled bioreactor vessel 12, pump mounting plate 14, and inline filter 17. The bioreactor vessel 12 is shown mounted on a tiltable tray 22 of the bioreactor platform 20. The bioreactor platform 20 may be a WAVE bioreactor, such as a Xuri™ cell expansion system, or another system having a mechanism for tilting the vessel 12 to a substantially upright position, i.e., to an angle of approximately 75 degrees from horizontal.

The pump mounting plate 14 is shown installed on a plurality of peristaltic pumps 15, e.g., Xuri™ W25 pumps, such that fluid lines 19 connected to the pump mounting plate 14 operatively contact pump heads of the peristaltic pumps 15. The pump mounting plate 14 further includes an inline filter 17, e.g., a tangential flow filter, which, is fluidly coupled to the bioreactor vessel 12 and a waste bag 19 via fluid lines and pumps. In embodiments, and as shown in FIG. 2, it is envisaged that the bioreactor vessel 12 will be a component of a preassembled kit that includes a mounting plate 14, filter 17, fluid lines, vessel 12 and waste bag 19.

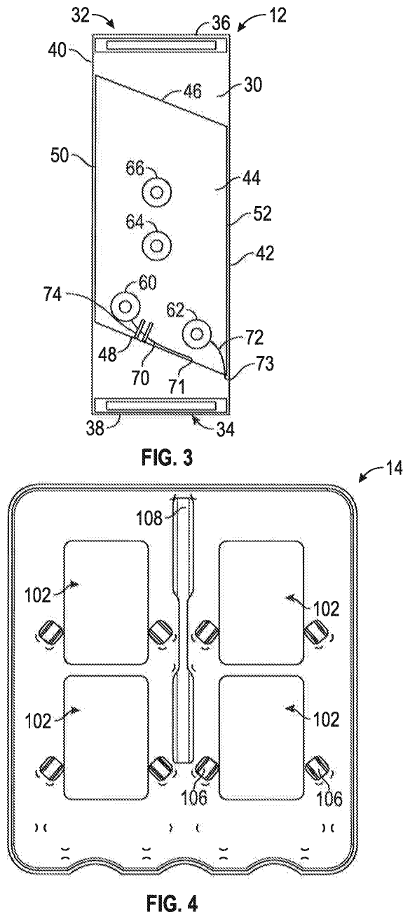

As will be discussed in greater detail below, the bioreactor vessel 12 includes a slanted seam/lower boundary 48, which, when the vessel is substantially upright, guides fluid in the vessel 12 toward an outlet port 62 having a dip tube 72 (FIG. 3). The slanted lower boundary 48, outlet port 62 and dip tube 72 allow fluid to be easily and quickly extracted for concentration and/or washing using pumps 15, a plurality of fluid lines, and filter 17. Moreover, the vessel 12 enables a closed bioprocessing system in which cells may be concentrated and/or washed without having to utilize a separate bioprocessing device.

Referring now to FIG. 3, in an embodiment, the bioreactor vessel 12 has a flexible, transparent body 30 with a substantially rectangular shape. The body 30 has an upper end 32 and a lower end 34, which are defined by top and bottom edge portions, 36 and 38, respectively. The upper end 32 is situated above the lower end 34 when the bioreactor vessel 12 is placed in a substantially upright position through a tiltable bioreactor platform or the like. The bioreactor vessel body 30 further includes first and second side edge portions 40, 42, respectively.

As shown, the body 30 also includes a hollow interior cavity 44 configured to receive biomaterials, e.g., T cells, for processing. The hollow interior cavity 44 is defined by an upper boundary 46, a lower boundary 48, and side boundaries, 50, 52. The interior cavity 44 includes a fluid inlet port 60 and a fluid outlet port 62, which, in certain embodiments, are proximate to the lower end 34 of the body 30, and may be substantially parallel to the lower boundary 48 in specific embodiments. In certain embodiments, the cavity 44 may also include gas inlet 64 and gas outlet 66 ports. The ports may be formed in the cavity 44 in a variety of ways, including welding them in place, and the ports may be barbed.

Importantly, as mentioned, the lower boundary 48 is slanted or angled downward toward the lower end 34 of the body 30. In embodiments, the lower boundary 48 is at an angle of about 45 degrees to about 75 degrees from vertical. In specific embodiments, the angle of the lower boundary 48 is about 62 degrees from vertical. In embodiments, the lower boundary 48 may be a welded seam between flexible polymeric sheets that define or form at least a portion of the interior cavity 44 of the body 30. In other embodiments, the lower boundary 48 may be a separate piece of material or structure from the interior cavity 44. Though the upper boundary 46 is also depicted as angled downward toward the lower end 34, it may be in a variety of orientations.

The fluid inlet port 60 and the fluid outlet port 62 include dip tubes which are positioned to maximize mixing during, for example, cell washing. More specifically, the inlet port 60 has an inlet dip tube 70, and the outlet port 62 has an outlet dip tube 72. As shown, the inlet dip tube 70 is substantially parallel to, and extends along the lower boundary 48 of the interior cavity 44 so that liquid flowing into the hollow interior cavity 44 through the inlet port 60 generates a vortex to facilitate mixing. The inlet dip tube 70 is maintained in proximity to the lower boundary 48 by one or more strips of film that are welded into the interior cavity. As will be appreciated, in certain embodiments, the inlet dip tube 70 may be secured in place through other means such as adhesives and the like. In embodiments, the inlet dip tube 70 extends a distance of 2″ to 6″ along the lower boundary 48 and is spaced approximately within about 1″ from the lower boundary 48.

Moreover, the distal end 71 of the inlet dip tube 70 must be spaced apart from the distal end 73 of the outlet dip tube 72 to prevent liquid shortcutting, i.e., when liquid entering the hollow interior cavity 44 via the inlet port 60 is immediately extracted out vessel 12 through the outlet port 62 without sufficient mixing. In embodiments, the distal end 71 of the inlet dip tube 70 is spaced apart from the distal end 73 of the outlet dip tube 72 at a distance of about 2″ to about 6″.

The distal end 73 outlet dip tube 72 is located proximate to an intersection of side boundary 52 of the interior cavity 44 and a portion of the lower boundary 48 that is closest to the lower end 34 of the vessel body 30. In embodiments, the distal end 73 of the outlet dip tube 72 contacts this intersection. The outlet dip tube 72 is in contact with, or in close proximity to, the lowest point of the interior cavity to ensure that a maximum volume of biomaterials, e.g., cells, are extractable from the interior cavity through the outlet port 62.

The bioreactor vessel may be a single use or multi-use bag and may be manufactured from a flexible plastic and the hollow cavity boundaries may be fused seams. In certain embodiments, the bioreactor vessel may have a rigid structure.

In a specific embodiment, the bioreactor vessel 12 is a flexible bag that is about 8″×22″ with the hollow interior cavity being substantially rhomboid in shape and having dimensions of about 7.5″×15.5.″ Moreover, in an embodiment, the inlet port dip tube 70 is about 6″ to about 8″ in length, and the outlet port dip tube 72 is about 3″ to about 5″ in length. As will be appreciated, however, the vessel and cavity may have a variety of shapes, sizes and configurations, and the ports may be in a variety of locations. Moreover, the length of the dip tubes may vary upon the location of the ports, as long as the distal ends of dip tubes are positioned to allow for functional mixing and extraction.

Turning now to FIG. 4, a pump mounting plate 14 according to an embodiment of the invention is depicted. In the figure, a user facing side of the pump mounting plate 14 is shown. In embodiments, the mounting plate 14 is manufactured from a transparent or semi-transparent material so that features, e.g., lights, on the pump modules may be visualized during use. As shown, the plate 14 includes a plurality of apertures 102, though which the pump heads pass allowing the plate 14 to be mounted on a plurality of peristaltic pumps. FIG. 4 depicts an embodiment that includes four apertures 102, but, as will be appreciated, other embodiments may include greater or fewer than four apertures.

In embodiments, the plate 14 is configured for mounting to peristaltic pump modules, each module including two pumps. Such modules may be operatively connectable, so that two modules may be stacked to scale to four pumps, e.g., the configuration depicted in FIG. 1.

The plate 14 further includes pump loop brackets 106, which are substantially U-shaped brackets located on the plate 14. The brackets 106 are sized and shaped to receive fluid lines and hold the fluid lines in a position such that they are in operative contact with the pump heads. As shown, each aperture 102 includes two brackets 106. In use, brackets 106 receive pump tubing sections of the fluid lines, e.g., silicone tubing, and hold the fluid lines in place via a press fit. The brackets 106 urge the pump tubing sections 21 into an arcuate path such that the pump tubing sections contact the pump heads allowing the pumps to function properly.

In embodiments, the fluid lines have multiple, interconnected sections of different materials. For example, the lines may have sections manufactured from a pharmaceutical grade thermoplastic elastomer, from PVC, and sections manufactured from silicone. In embodiments, the fluid lines have pump tubing sections 21, which are manufactured from silicone or functionally similar material. In specific embodiments, the fluid lines include 6″ silicone pump tubing sections 21, though as will be appreciated, pump tubing sections may vary in length, and be manufactured from a variety of materials, as long as the pump tubing sections 21 sufficiently engage a pump head to allow for proper pump functioning.

The pump mounting plate 14 further includes a filter bracket 108. The filter bracket 108 is sized and shaped to receive a tangential flow filter, e.g., a hollow fiber filter, and hold the filter in place via a press or snap fit.

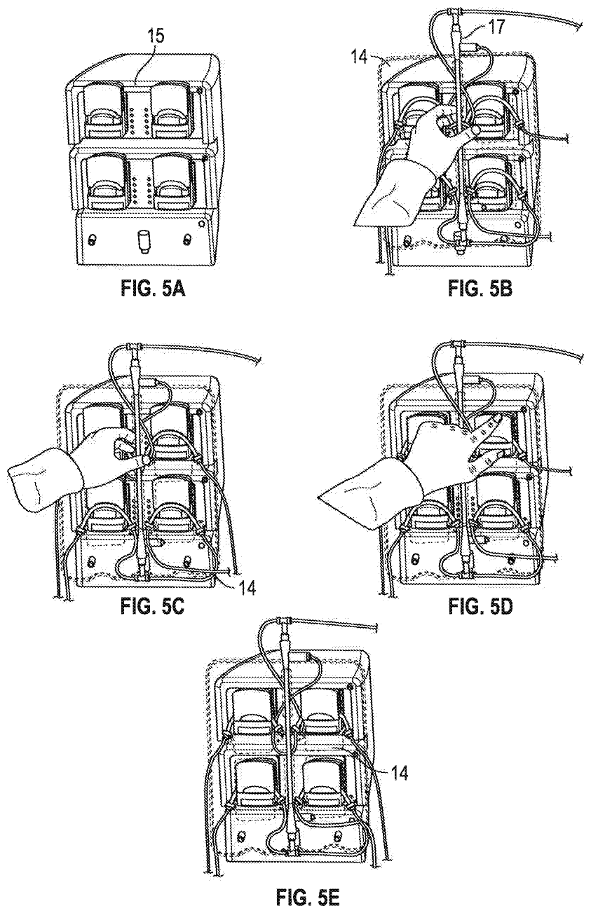

Referring now to FIGS. 5A-5E, an embodiment of pump mounting plate 14 is depicted being installed on a plurality of peristaltic pumps 15. In an embodiment, the pump mounting plate 14, shown in dashed lines, is preloaded with four pump head engaging fluid lines, two of which are connected to the outlet port of the bioreactor vessel 12 and waste bag 19 (FIG. 2), and two or which are connectable to inoculum/buffer bags and media bags via, for example, sterile tube fusing. The lines may all also be operatively connected to the filter via T-connectors.

To install the mounting plate 14 on a plurality of pumps, a user first opens the covers of the pump heads (FIG. 5A). Then, the pump tubing sections of the fluid lines are aligned with the pump heads (FIG. 5B) and the mounting plate 14 is pushed into place (FIG. 5C). The pump head covers are then closed (FIG. 5D) and the user may then check the functionality of the pumps. As will be appreciated, by preloading the fluid lines on the pump mounting plate 14 and having the lines pre-attached to the vessel 12 and waste bag 19, embodiments of the system provide an ease of installation, use and manufacture of cell therapies. Moreover, in certain embodiments, each of the apertures is labeled so that a user can quickly identify the function of each fluid line connected thereto to facilitate attachment to the appropriate bag.

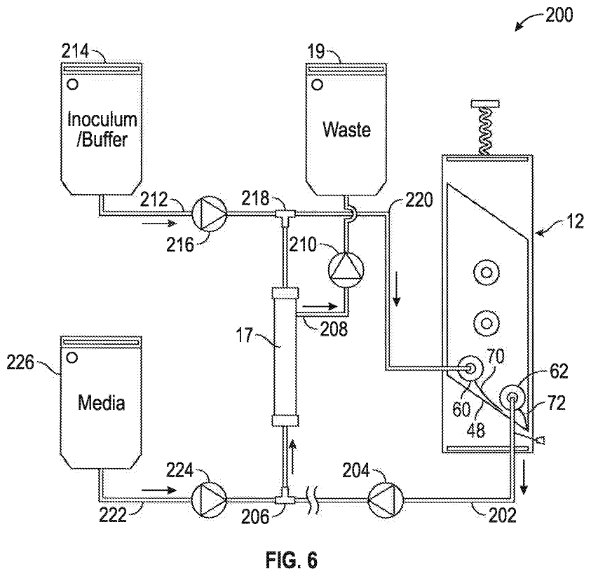

Referring to FIG. 6, a flow path of a bioprocessing system 200 according to an embodiment is depicted. As shown, the bioreactor vessel 12 has an outlet fluid line 202 that is connected to the outlet port 62. The outlet fluid line 202, in turn, engages a first pump 204 and is connected to the filter 17 via a T-connector 206, or a functionally similar valve. The filter 17 includes a waste fluid line 208 which engages a second pump 210 and feeds into a waste bag 19.

The system 200 further includes an inoculum/buffer fluid line 212 which is connected to an inoculum or buffer bag 214 and engages a third pump 216. The inoculum/buffer fluid line 212 connects to a T-connector 218 of the filter 17. The T-connector 218 is, in turn, connected to an inlet fluid line 220 for fluid return to the bioreactor vessel 12 via the inlet port 60 and inlet dip tube 70.

The system 200 also includes a media fluid line 222 for media replenishment. The media fluid line 220 engages a fourth pump 224 and T-connector 206. The media fluid line 220 is connectable to a media bag 226.

In embodiments, the vessel 12 may be connected to a load cell 228. Moreover, embodiments further include a clave port 230 located on, for example, the outlet fluid line 202, to facilitate sampling to assess, for example, cell population density.

As will be appreciated, system 200 is configured for use with the mounting plate 14, which has been omitted from the schematic in the figure. Similarly, the functionality of system 200 discussed above is facilitated by mounting the bioreactor vessel 12 on a tilting tray of a bioreactor platform and tilting the vessel 12 to a substantially upright position. The platform and substantially upright position are not depicted in the figure.

In embodiments, a closed bioprocessing system is provided in which cell concentration and/or washing may be accomplished without the need for a separate bioprocessing device, e.g., vessel, centrifuge, or the like. In aspects, this functionality achieved through the flow path architecture shown in FIG. 6.

More specifically, for concentration, the bioreactor vessel 12 is placed in a substantially upright position and the first pump 204 is activated to draw the cell suspension out of the bioreactor vessel 12 via the outlet port 62 and outlet dip tube 72, through the outlet fluid line 202 and T-connector 206, and into the filter 17. The second pump 210, i.e., a permeate pump, is started to draw water out of the cell suspension and into the waste bag 19 via the waste fluid line 208. Cells are returned back into the vessel 12 through the inlet fluid line 220 and inlet port 60 and inlet dip tube 70. During concentration, the second and fourth pumps 216, 224, respectively, are closed and function as pinch valves.

As will be appreciated, this process is performed until a desired concentration is achieved, e.g., a reduction of from 250 ml of cell suspension down to 50 ml of suspension. Once the desired concentration is accomplished, the cells may be washed or otherwise processed.

With respect to washing, pump 204, i.e. the main loop pump, is activated to draw the cell suspension out of the bioreactor vessel 12 via the outlet port 62 and outlet dip tube 72, through the outlet fluid line 202 and T-connector 206, and into the filter 17. In embodiments, pump 204 circulates fluid through the filter 17 while maintaining an amount of shear force whereby the cells are not pulled into and caught within porous permeate ‘mi tunnels’ within the filter 17.

With pump 204 still active wash buffer from inoculum/buffer bag 214 is pumped via pump 216 into the inlet port 60 and inlet dip tube 70 of the bioreactor vessel 12 via the inoculum/buffer fluid line 212, through T-connector 218 and fluid line 220. Following activation of pumps 204 and 216, pump 210, i.e., a permeate pump, is activated to draw water out of the cell suspension and into the waste bag 19 via the waste fluid line 208. Cells are returned back into the vessel 12 through the inlet fluid line 220 where they mix with wash buffer. In embodiments, pumps 216 and 208 are operated at the same flow rate to maintain equilibrium, i.e., volume in is equal to volume out so no further concentration or dilution occurs, while the cells are washed with the fresh incoming wash buffer.

As will be appreciated, cells may be washed and/or concentrated after activation or genetic modification, in addition to expansion, in the vessel 12. Moreover, once the concentrated and/or washed cells are returned to the bioreactor vessel, the volume of cell suspension may be reconstituted with media to obtain a desired cell density and the culture contents may then be placed in a new bioreactor vessel where they can be further expanded.

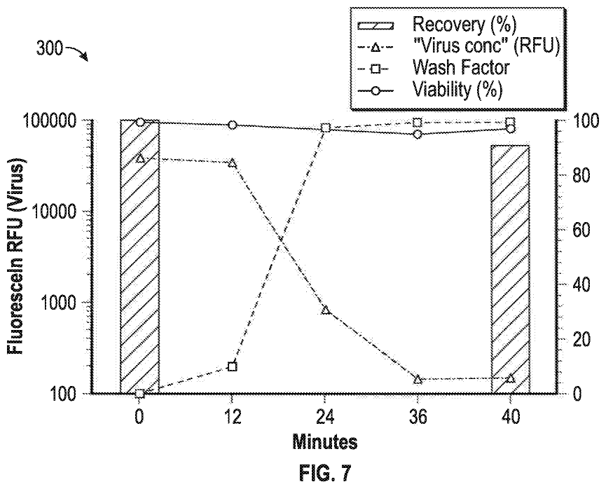

Referring now to FIG. 7, a graph 300 is provided that shows the efficacy of an embodiment of the present invention in concentrating and washing cells. In particular, this graph 300 is representative of the efficiency of an embodiment of the present invention in both concentration of cell suspension, i.e., de-watering and vector washing.

To simulate the required washing step to remove undesirable contents such as viral vectors used in cell engineering, fluorescein dye was added to a cell suspension (150 nM). Cell suspension was concentrated prior to washing and both unit operations leverage the incorporated tangential flow filter to remove undesired contents. Samples were collected periodically throughout process and analyzed for fluorescence. The surrogate fluorescein concentration is represented by the “Virus cone” (RFU) plot using a log scale. The concentration of fluorescein measured in Relative Fluorescence Units (RFU) scaled on the left axis decreases dramatically over 36 minutes of processing time. This washing is represented by the “Wash factor” line plot. The test results show in this figure illustrated a 2-log reduction in fluorescein after 40 mins processing time.

The percentage for cell viability (line), wash factor, as well as cell recovery (bar) post processing are shown on the right axis. The cell viability remains at nearly 100% throughout the wash process from Time=0 minutes to Time=40 minutes, Similarly, a 90% cell recovery is achieved. Both recovery and viability values demonstrate a robust process that is also a highly efficient.

Formula for calculating Wash factor:

Wash factor=(Original Virus Counts−current counts)*100%/Original virus counts.

Using fluorescein as the surrogate,

Virus counts=RFU concentration*current processing volume

At the beginning, no virus was washed, so the washing factor is 0

If wash factor=90%, then, it's 1 log wash

If wash factor=99%, then it's 2 Log wash

If wash factor is 99.9%, then it's 3 Log wash

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

Additionally, while the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, terms such as “first,” “second,” “third,” “upper,” “lower,” “bottom,” “top,” etc. are used merely as labels, and are not intended to impose numerical or positional requirements on their objects. Further, the limitations of the following claims are not written in means-plus-function format are not intended to be interpreted as such, unless and until such claim limitations expressly use the phrase “means for” followed by a statement of function void of further structure.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. A bioreactor vessel, comprising:
a body having upper and lower ends;
a hollow interior cavity formed in the body, the interior cavity located between the upper and lower ends, the interior cavity being configured to receive biomaterials for processing;
wherein the interior cavity has a lower boundary that is angled toward the lower end of the body such that the vessel may be tilted to allow biomaterials within the interior cavity to be extracted and concentrated and/or washed without the need for a separate bioprocessing device.

2. The bioreactor vessel of claim 1, wherein the lower boundary is at an angle of about 45 to about 75 degrees.

3. The bioreactor vessel of claim 2, wherein the lower boundary is at an angle of about 62 degrees.

4. The bioreactor vessel of claim 1, wherein the interior cavity includes a fluid outlet port located adjacent to a portion of the lower boundary that is proximate the lower end of the vessel body.

5. The bioreactor vessel of claim 4, wherein the fluid outlet port includes an outlet dip tube.

6. The bioreactor vessel of claim 5, wherein a distalend of the outlet dip tube is located proximate to an intersection of a side boundary of the interior cavity and a portion of the lower boundary that is closest to the lower end of the vessel body.

7. The bioreactor vessel of claim 1, wherein the interior cavity further includes a fluid inlet port located adjacent to a portion of the lower boundary.

8. The bioreactor vessel of claim 7, wherein the fluid inlet port includes an inlet dip tube.

9. The bioreactor vessel of claim 8, wherein at least a portion of the inlet dip tube is maintained in close proximity to the lower boundary of the interior cavity.

10. The bioreactor vessel of claim 1, wherein the lower boundary is a welded seam.

11. The bioreactor vessel of claim 1, wherein the vessel is a flexible, single use cell processing bag.

12. A bioprocessing system comprising:
a bioreactor vessel having an interior cavity with a lower boundary at an angle of about 45 to about 75 degrees, the vessel configured for use with a tiltable bioreactor platform;
a pump mounting plate configured to engage a plurality of peristaltic pumps such that a plurality of fluid lines connectable to the bioreactor vessel operatively contact pump heads; and
wherein upon tilting the bioreactor vessel to a substantially upright position, cells may be extracted from the bioreactor vessel and concentrated and/or washed, with the aid of the peristaltic pumps and an inline tangential flow filter, such that the cell concentration and/or washing can be accomplished without the need for a separate bioprocessing device.

13. The bioprocessing system of claim 12 further comprising:
a waste receptacle fluidly connected to the filter via at least one fluid line.

14. The bioprocessing system of claim 12 further comprising:
a wash buffer bag fluidly connected to the vessel via at least one fluid line.

15. The bioprocessing system of claim 12 further comprising:
a media bag fluidly connected to the vessel via at least one fluid line.

16. The bioprocessing system of claim 12 further comprising:
a fluid outlet port located adjacent to a portion of the lower boundary that is proximate to a lower end of the bioreactor vessel, the fluid outlet port having a dip tube.

17. The bioprocessing system of claim 16, wherein a distal end of the dip tube is located proximate to an intersection of a side boundary of the interior cavity and a portion of the lower boundary that is closest to the lower end of the vessel body.

18. The bioprocessing system of claim 12, wherein the vessel is a flexible, single use cell processing bag.

19. The bioprocessing system of claim 12, wherein the pump mounting plate has four apertures configured to engage four peristaltic pump heads.

20. The bioprocessing system of claim 12, wherein the pump mounting plate includes a plurality of pump loop brackets configured to receive pump tubing sections and position the fluid lines so that they are in operative contact with the peristaltic pumps.

21. A method of bioprocessing, comprising:
processing cells in a bioreactor vessel having an interior cavity i a lower boundary at an angle of about 45 to about 75 degrees;
tilting the bioreactor vessel to substantially upright position so that the cells may be extracted from the vessel;
extracting cells from the bioreactor vessel;
concentrating and/or washing the extracted cells; and
wherein the steps of concentrating and/or washing the extracted cells are carried out without the need for a separate bioprocessing device.

22. The method of claim 21, wherein the bioreactor vessel includes a fluid outlet port located adjacent to a portion of the lower boundary that is proximate a lower end of the bioreactor vessel, the fluid outlet port having a dip tube; and
wherein the fluid outlet port and dip tube facilitate extraction of the cells from the vessel.

23. The method of claim 22, wherein a distal end of the dip tube is located proximate to an intersection of a side boundary of the interior cavity and a portion of the lower boundary that is closest to the lower end of the vessel body.

24. The method of claim 21, wherein:
the cells are concentrated and/or washed via a tangential flow process utilizing a filter.

25. The method of claim 24, wherein the filter is a hollow fiber filter.

26. The method of claim 21 further comprising the step of:
operatively connecting the bioreactor vessel to at least one peristaltic pump via a pump mounting plate configured to engage the pump such that fluid lines connected to the vessel are in operative contact with the pump.

27. The method of claim 21, wherein the bioreactor vessel is tilted when the tray of a wave bioreactor platform is tilted.

28. The method of claim 21, wherein the step of processing the cells comprises:
activating the cells; and/or
genetic modification of the cells.

29. The method of claim. 21 further comprising the steps of:
returning the concentrated and/or washed cells to the bioreactor vessel; and
adding media to obtain desired cell density before transferring the cells to a new bioreactor vessel.
expanding the concentrated and/or washed cells in the new bioreactor vessel.

30. A pump mounting plate for operatively connecting a bioreactor vessel to at least one peristaltic pump, the pump mounting plate comprising:
at least one aperture through which a peristaltic pump can extend facilitating installation of the pump mounting plate to the peristaltic pump;
a plurality of pump loop brackets configured to receive fluid lines connected to the vessel and position the fluid lines so that they are in operative contact with pump heads of the peristaltic pumps; and
a filter bracket configured to removably secure a filter to the pump mounting plate; and
wherein the pump mounting plate is configured to be preloaded with fluid lines, to provide an ease of installation and use of the vessel for cell processing.
